(19) United States
(12) Patent Application Publication
(10) Pub. No.: US 2021/0365876 A1
(43) Pub. Date: Nov. 25, 2021

(54) SYSTEMS, METHODS, AND APPARATUSES FOR IMPLEMENTING MACHINE LEARNING MODEL TRAINING AND DEPLOYMENT FOR PREDICTIVE INVENTORY PURCHASING DATABASE

(71) Applicants: CARY JAMES BREESE, LAGUNA NIGUEL, CA (US); SUMEET SHEOKAND, SUNNYVALE, CA (US)

(72) Inventors: CARY JAMES BREESE, LAGUNA NIGUEL, CA (US); SUMEET SHEOKAND, SUNNYVALE, CA (US)

(21) Appl. No.: 17/243,575

(22) Filed: Apr. 29, 2021

Related U.S. Application Data
(63) Continuation-in-part of application No. 16/249,899, filed on Jan. 17, 2019.
(60) Provisional application No. 62/618,154, filed on Jan. 17, 2018.

(51) Int. Cl. G06Q 10/08; G06N 20/00; G16H 20/13

(52) U.S. Cl. CPC G06Q 10/087; G16H 20/13; G06N 20/00

(57) ABSTRACT

In one aspect, a computerized method for implementing machine learning model training and deployment for predictive inventory purchasing database includes the step of providing a predictive inventory purchasing database. The method includes providing a machine-learning platform coupled with the predictive inventory purchasing database. The method includes retrieving a set of training data as input to the machine learning platform. The method includes based on the set of training data, generating a predictive inventory purchasing model with the machine learning platform. The method includes using the predictive inventory purchasing model to automatically implement inventory purchasing.

### CROSS-REFERENCE TO RELATED APPLICATIONS

This application is a continuation in part of and claims priority to U.S. patent application Ser. No. 16/249,899, filed on Jan. 17, 2019 and titled METHOD AND SYSTEM OF AN AUTOMATED MEDICATION DISPENSING AND DELIVERY SYSTEM. This application is hereby incorporated by reference in its entirety.

U.S. patent application Ser. No. 16/249,899 application claims priority from U.S. Provisional Patent Application No. 62618154, filed on 17 Jan. 2018 and titled METHOD AND SYSTEM OF AN AUTOMATED MEDICATION DISPENSING AND DELIVERY SYSTEM. This application is hereby incorporated by reference in its entirety.

### FIELD OF THE INVENTION

The invention is in the field of automated system optimization and more specifically to a method, system, and apparatus for implementing machine learning model training and deployment for predictive inventory purchasing database, according to some embodiments.

### DESCRIPTION OF THE RELATED ART

There is a trend in pharmacy to switch to automated fill of prescription medications from remote, central locations that combine and integrate delivery logistics and pharmacy processing in order to reduce costs and inefficiencies and increase convenience and reliability to patients. However, efficient remote, central fill automation requires integration of complex pharmacy processes, efficient communication with remote patients, intelligent communication with physicians and health insurance providers and optimized delivery logistics. Accordingly, improvements to automated medication dispensing, processing and delivery are desired.

### BRIEF SUMMARY OF THE INVENTION

In one aspect, a computerized method for implementing machine learning model training and deployment for predictive inventory purchasing database includes the step of providing a predictive inventory purchasing database. The method includes providing a machine-learning platform coupled with the predictive inventory purchasing database. The method includes retrieving a set of training data as input to the machine learning platform. The method includes based on the set of training data, generating a predictive inventory purchasing model with the machine learning platform. The method includes using the predictive inventory purchasing model to automatically implement inventory purchasing.

### BRIEF DESCRIPTION OF THE DRAWINGS

The present application can be best understood by reference to the following description taken in conjunction with the accompanying figures, in which like parts may be referred to by like numerals.

FIG. 1 illustrates an example automated medication dispensing and delivery system, according to some embodiments.

FIG. 2 depicts an exemplary computing system that can be configured to perform any one of the processes provided herein.

FIG. 3 is a block diagram of a sample computing environment that can be utilized to implement various embodiments.

FIG. 4 illustrates optimizing medication delivery using opportunistic refill opportunities in a specified geographic region, according to some embodiments.

FIG. 5 illustrates an example process for optimization of medicine purchase prices, according to some embodiments.

FIG. 6 illustrates end-to-end robotic dispensing system using voice activation, according to some embodiments.

FIG. 7 illustrates an example process for implementing machine learning model training and deployment for predictive inventory purchasing database, according to some embodiments.

The Figures described above are a representative set, and are not an exhaustive with respect to embodying the invention.

### DETAILED DESCRIPTION

Disclosed are a method and system of an automated medication dispensing and delivery system. Although the present embodiments have been described with reference to specific example embodiments, it can be evident that various modifications and changes may be made to these embodiments without departing from the broader spirit and scope of the particular example embodiment.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, attendee selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labelled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### EXAMPLE DEFINITIONS

Application programming interface (API) can specify how software components of various systems interact with each other.

Cloud computing can involve deploying groups of remote servers and/or software networks that allow centralized data storage and online access to computer services or resources. These groups of remote serves and/or software networks can be a collection of remote computing services.

Machine learning can include the construction and study of systems that can learn from data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning.

Virtual assistant (e.g. an intelligent personal assistant) can be a software agent capable of voice interaction that can perform tasks or services for an individual.

### Example Systems

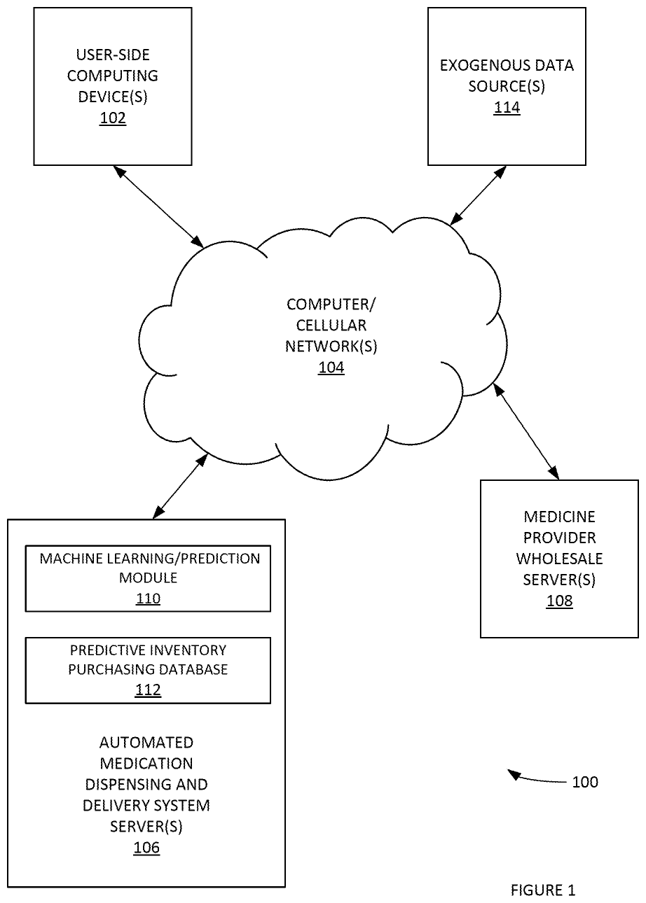

FIG. 1 illustrates an example automated medication dispensing and delivery system 100, according to some embodiments. System 100 can be designed to automatically provide various medication deliveries to users at a specified location (e.g. home, office, hotel, hospital, doctor's office, school, etc.). medicine (re)filling, review, and delivery can be managed by automated medication dispensing and delivery system server(s) 106. Users can upload various information to automated medication dispensing and delivery system server(s) 106 via user-side computing device(s) 102. Example information can include, inter alia: user's pharmaceutical prescriptions, other medicines user is ordering, etc. Medicine can be prescription drugs, non-prescription drugs, compounded medicine services, medical devices, other medical services, etc. Non-medicine/medical products (e.g. groceries, etc.) can also be delivered with system 100. Users can interact with an automated system using text messages from their phones, or a chat service within the NowRx smartphone application. Through this interaction, an automated system can interpret the user's refill requests, their chosen delivery time and location, amongst other operations. Once confirmed, this information is forwarded to the fulfilling system. Automated medication dispensing and delivery system server(s) 106 can manage the refilling and delivery of medications to customers at regular intervals, usually once per month on or about the same day of the month (e.g. an ordinary refill queue). Automated medication dispensing and delivery system server(s) 106 can also identify and manage opportunistic refills (e.g. see process 400 infra).

Automated medication dispensing and delivery system server(s) 106 can remotely manage automated medication dispensing systems that are onsite at various pharmacies. Automated medication dispensing and delivery system server(s) 106 can store medicine (re)fill information of customers in a datastore.

Automated medication dispensing and delivery system server(s) 106 can implemented processes 400, 500, and/or 600 provided infra. Automated medication dispensing and delivery system server(s) 106 can include various functionalities such as, inter alia: machine-learning systems, mapping and vehicle routing systems, route optimization systems, database managers, calculators, purchasing systems, API's, email servers, text-message servers, etc. It is noted that various server-side functionalities and systems can be implemented in a cloud-computing environment in some example embodiments.

Automated medication dispensing and delivery system server(s) 106 can order medicines and/or other medical supplies from medicine wholesale server(s) 108. For example, automated medication dispensing and delivery system server(s) 106 can manage an automated, optimized purchasing using wholesale spot pricing process. It is noted the drug prices on the wholesale market can have a high degree of variability. Each drug compound is available from multiple manufacturers, sold by a variety of wholesalers, each with their own individual price and those prices vary daily. Automated medication dispensing and delivery system server(s) 106 can automatically analyze prices across wholesalers, manufactures and drug formulation, and identify the lowest price each day, thereby optimizing wholesale purchases. The automation system further tracks historical prices and available quantities at various wholesalers. Combined with current inventory levels at any given pharmacy location and the predicted medication usage in an evaluated time period, the automated system uses aspects of machine learning to build an artificial intelligence system that would decide to proactively buy or delay purchasing medicines from the wholesaler.

Automated medication dispensing and delivery system server(s) 106 can include machine learning/prediction module 110. Machine learning/prediction module 110 can perform the machine-learning functions of process 700 discussed infra. Machine learning/prediction module 110 can obtain data from training data set(s) such as those stored in predictive inventory purchasing database 112. Machine learning/prediction module 110 can create a predictive inventory purchasing model from training data from predictive inventory purchasing database 112. Machine learning/prediction module 110 can obtain data from the other modules of automated medication dispensing and delivery system server(s) 106. Machine learning/prediction module 110 can implement machine learning algorithms on the data and obtain patterns and inference from said data. Machine learning/prediction module 110 can enable the various modules of automated medication dispensing and delivery system server(s) 106 to perform specific tasks without using explicit instructions. Machine learning/prediction module 110 can make predictions regarding future pharmaceutical transactions. These predictions can be used to implement a predictive inventory purchasing model to automatically implement inventory purchasing.

Machine learning/prediction module 110 can utilize machine learning algorithms to recommend and/or optimize various automated inventory services.

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning. Random forests (RF) (e.g. random decision forests) are an ensemble learning method for classification, regression, and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, that is a set of examples used to fit the parameters (e.g. weights of connections between neurons in artificial neural networks) of the model. The model (e.g. a neural net or a naive Bayes classifier) is trained on the training dataset using a supervised learning method (e.g. gradient descent or stochastic gradient descent). In practice, the training dataset often consist of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g. the number of hidden units in a neural network). Validation datasets can be used for regularization by early stopping: stop training when the error on the validation dataset increases, as this is a sign of overfitting to the training dataset. This procedure is complicated in practice by the fact that the validation dataset's error may fluctuate during training, producing multiple local minima. This complication has led to the creation of many ad-hoc rules for deciding when overfitting has truly begun. Finally, the test dataset is a dataset used to provide an unbiased evaluation of a final model fit on the training dataset. If the data in the test dataset has never been used in training (e.g. in cross-validation), the test dataset is also called a holdout dataset.

Automated medication dispensing and delivery system server(s) 106 can leverage exogenous data sources to obtain training data (e.g. historical purchasing and sales transactions, etc.) that may not be available internally.

Automated medication dispensing and delivery system server(s) 106 can manage an opportunistic refill processing based on geographic input process. The opportunistic refill processing can use geographic location of customer location to opportunistically process and deliver medications several days early when it is possible to optimize delivery efficiency. Delivery efficiency can be measured by a number of refills delivered to same neighborhood on same day. For example, if a new or refill prescription request is scheduled for delivery on a specified day to a certain neighborhood, automated medication dispensing and delivery system server(s) 106 can scan the ordinary refill queue for the any refills destined for the same neighborhood albeit several days in the future as an opportunistic refill opportunity. These opportunistic refills can be processed a specified number of days early (e.g. two days early, three days early, one week early, etc.) in order to maximize a preset day's deliveries to a geographic region within a nearby specified distance of other customers in the ordinary refill queue. As a corollary process, automated medication dispensing and delivery system server(s) 106 can also modify the priority of prescriptions processed during the day to optimize route creation.

Automated medication dispensing and delivery system server(s) 106 can maintain a medication adherence index. The medication adherence index can be implemented various data values for, inter alia: patient demographics, physician identity, disease state, drug characteristics for medication adherence ranking and/or intervention statistics. The index can be used as a parameter in various algorithms provided herein. Additionally, the index can be used by health-care providers to determine a follow-up rate and/or other medication adherence strategies.

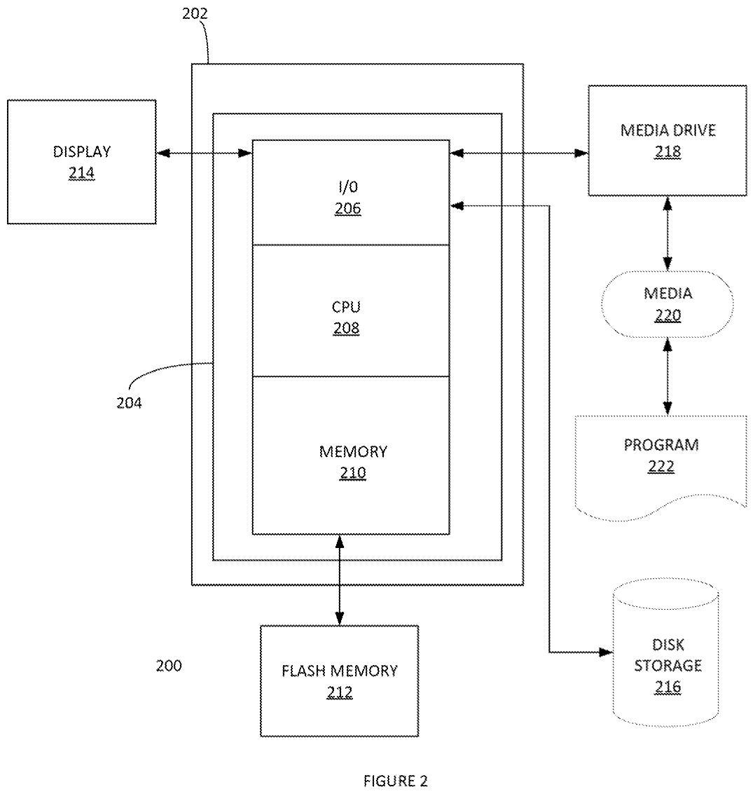

FIG. 2 depicts an exemplary computing system 200 that can be configured to perform any one of the processes provided herein. In this context, computing system 200 may include, for example, a processor, memory, storage, and I/O devices (e.g., monitor, keyboard, disk drive, Internet connection, etc.). However, computing system 200 may include circuitry or other specialized hardware for carrying out some or all aspects of the processes. In some operational settings, computing system 200 may be configured as a system that includes one or more units, each of which is configured to carry out some aspects of the processes either in software, hardware, or some combination thereof.

FIG. 2 depicts computing system 200 with a number of components that may be used to perform any of the processes described herein. The main system 202 includes a mother—board 204 having an I/O section 206, one or more central processing units (CPU) 208, and a memory section 210, which may have a flash memory card 212 related to it. The I/O section 206 can be connected to a display 214, a keyboard and/or other user input (not shown), a disk storage unit 216, and a media drive unit 218. The media drive unit 218 can read/write a computer-readable medium 220, which can include programs 222 and/or data. In some embodiments, parts or all of system 200 can be implemented in a cloud-computing environment.

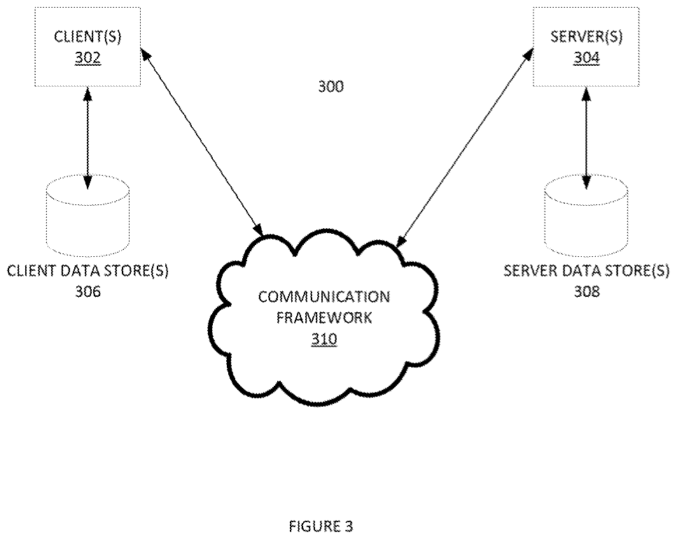

FIG. 3 is a block diagram of a sample computing environment 300 that can be utilized to implement various embodiments. The system 300 further illustrates a system that includes one or more client(s) 302. The client(s) 302 can be hardware and/or software (e.g., threads, processes, computing devices). The system 300 also includes one or more server(s) 304. The server(s) 304 can also be hardware and/or software (e.g., threads, processes, computing devices). One possible communication between a client 302 and a server 304 may be in the form of a data packet adapted to be transmitted between two or more computer processes. The system 300 includes a communication framework 310 that can be employed to facilitate communications between the client(s) 302 and the server(s) 304. The client(s) 302 are connected to one or more client data store(s) 306 that can be employed to store information local to the client(s) 302. Similarly, the server(s) 304 are connected to one or more server data store(s) 308 that can be employed to store information local to the server(s) 304. In some embodiments, system 300 can instead be a collection of remote computing services constituting a cloud-computing platform.

### Example Processes

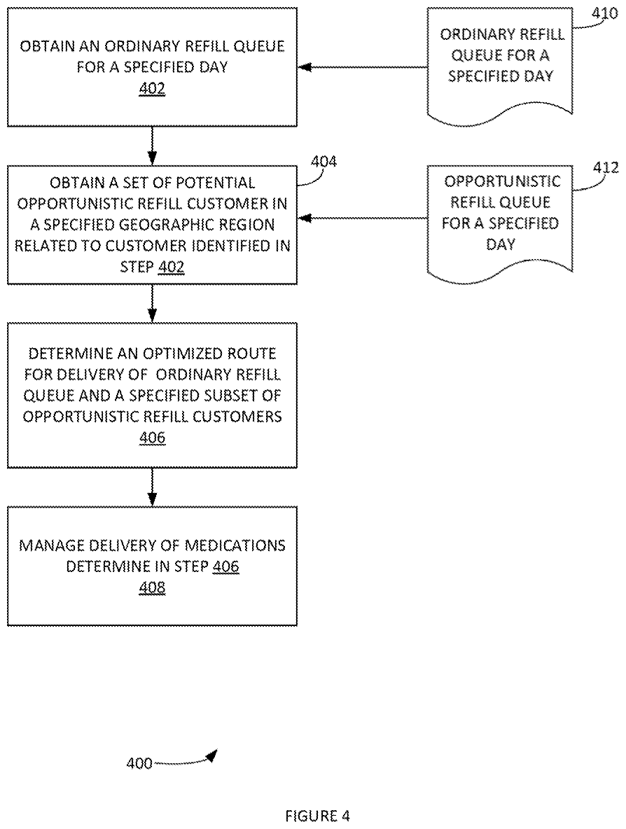

FIG. 4 illustrates optimizing medication delivery using opportunistic refill opportunities in a specified geographic region, according to some embodiments. In step 402, process 400 can obtain an ordinary refill queue for a specified day 410. In step 404 process 400 can obtain a set of potential opportunistic refill customers 412 in a specified geographic region related to customer identified in step 402. In step 408, process 400 can determine an optimized route for delivery of ordinary refill queue and a specified subset of opportunistic refill customers. In step 408, process 400 can manage delivery of medications determine in step 406.

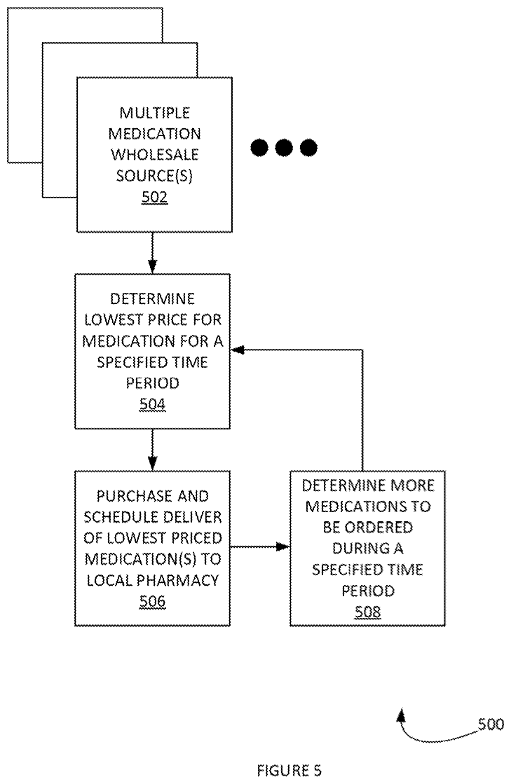

FIG. 5 illustrates an example process 500 for optimization of medicine purchase prices, according to some embodiments. In step 502, process 500 can communicate with multiple medication wholesale source(s). In step 504, process 500 can determine lowest price for medication for a specified time period. In step 506, process 500 can purchase and schedule deliver of lowest priced medication(s) to local pharmacy. In step 508, process 500 can determine more medications to be ordered during a specified time period.

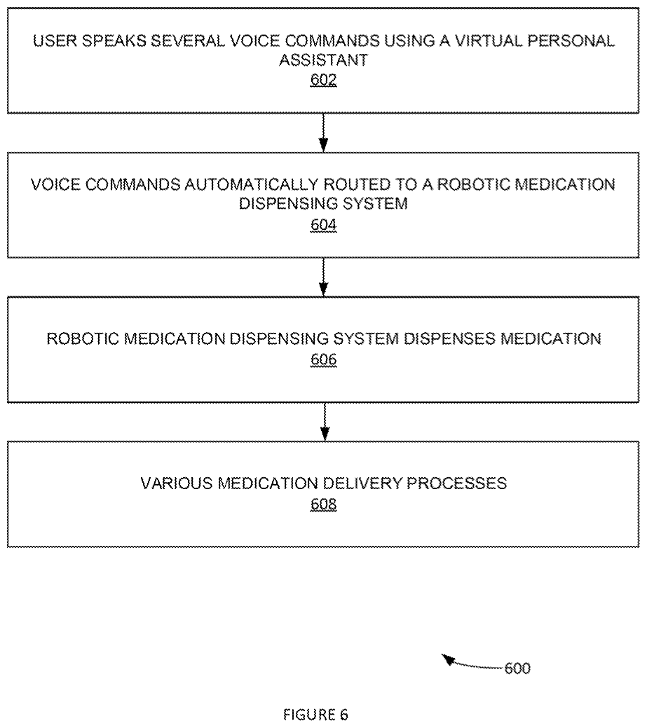

FIG. 6 illustrates end-to-end robotic dispensing system using voice activation, according to some embodiments. Process 600 can interface with an industry robotic dispensing system.

In step 602, process 600 can obtain several voice commands from a user using a virtual personal assistant. The voice commands can include information about medication refills. In step 604, the voice commands can be automatically routed to a robotic medication dispensing system. In step 606, process 600 can manage a robotic medication dispensing system that dispenses the medication. In step 606, process 600 can then various medication delivery processes such as those provided herein. Process 600 can also enable voice-activated refill requests and transfers from other pharmacies.

For example, a user can speak several voice commands using a virtual personal assistant, such as GOOGLE HOME ASSISTANT®, AMAZON ALEXA® or APPLE SIRI®. Using an Internet Protocol, the voice commands can be automatically routed to a commercially available machine that performs robotic medication filling (e.g. sort, count, bottle, label and cap). Process 600 can include the communication mechanism between commercially available virtual assistants and commercially available robotic dispensing systems.

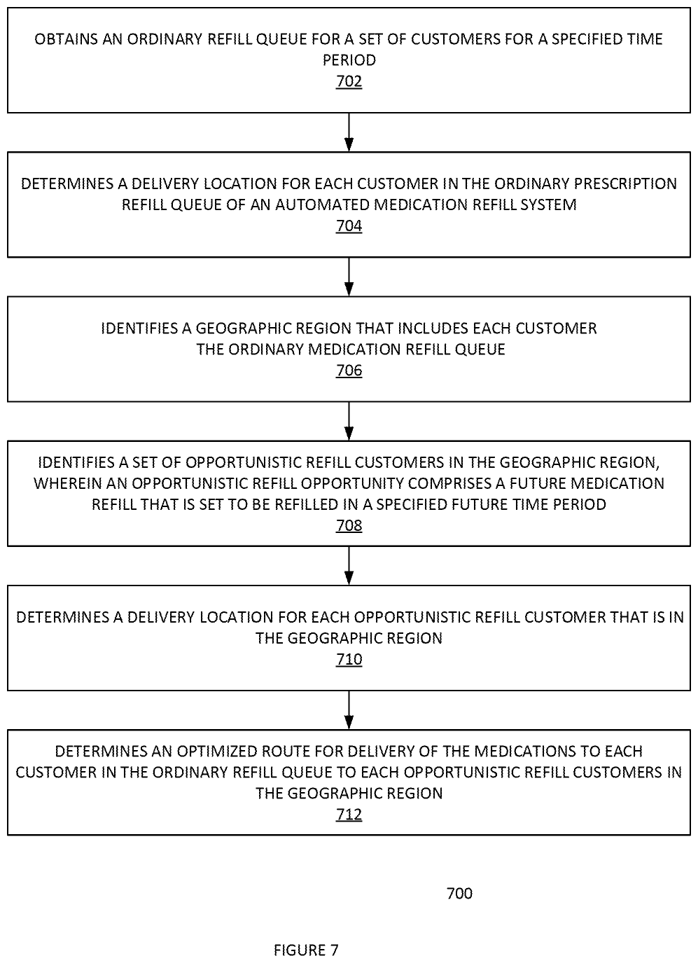

FIG. 7 illustrates an example process 700 for implementing machine learning model training and deployment for predictive inventory purchasing database, according to some embodiments. In step 702, process 700 provides a predictive inventory purchasing database. A machine-learning platform coupled with the predictive inventory purchasing database can be provided. This can be implemented by machine learning/prediction module 110 discussed supra.

In step 704, process 700 retrieves a set of training data as input to the machine learning platform. The training data includes historical purchasing and sales transactions, each of the transactions specifying specific characteristics upon which to make predictions of future purchasing requirements to optimize several pre-defined constraints.

It is noted that the multiple transactions within the training data can include any one or more of, inter alia:

A set of product factors that include, inter alia: a medication identifier, product identifier, medication schedule form of medication (e.g. tablet, pill, liquid, gel, etc.), price;

A set of date characteristics that include, inter alia: calendar date medication was dispensed national holiday schedule day of weekday, day of month, month of year time of day;

A quantity of medication identifier dispensed;

A pharmacy location where medication is dispensed from;

An actual refill remaining on prescription at time of fill;

An expected number of refills remaining;

A set of factors affecting expected general customer behavior that include, inter alia: a holiday calendar schedule, a weather forecast, a month of year, a day of week, etc.;

A set of factors affecting an Expected Individual Customer Behavior that include, inter alia: allergies age gender socioeconomic factors, etc.;

A set of historical inventory patterns by medication identifier that includes, inter alia: an out-of-stock occurrences degree of overstocking expiration timeline, etc.; and

In step 706, process 700, based on training data, generates a predictive inventory purchasing model with the machine learning platform.

Optionally, in step 708, process 700, periodically validates and updates the predictive inventory purchasing model with an updated version of the predictive inventory purchasing database. In step 710, process 700 uses the predictive inventory purchasing model to automatically implement inventory purchasing.

### B. Conclusion

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A computerized method for implementing machine learning model training and deployment for predictive inventory purchasing database comprising:
providing a predictive inventory purchasing database;
providing a machine-learning platform coupled with the predictive inventory purchasing database;
retrieving a set of training data as input to the machine learning platform;
based on the set of training data, generating a predictive inventory purchasing model with the machine learning platform; and
using the predictive inventory purchasing model to automatically implement inventory purchasing.

2. The computerized method of claim 1 further comprising:
providing an updated version of the predictive inventory purchasing database.

3. The computerized method of claim 1 further comprising:
periodically validating and updating the predictive inventory purchasing model with the updated version of the predictive inventory purchasing database.

4. The computerized method of claim 1, wherein the training data comprises a set of historical purchasing and sales transactions.

5. The computerized method of claim 4, wherein each of the transactions specifies a set of specific characteristics upon which to make predictions of future purchasing requirements to optimize a set of pre-defined constraints.

6. The computerized method of claim 5, wherein the set of historical purchasing and sales transactions comprises a set of product factors.

7. The computerized method of claim 6, wherein the set of product factors comprises a medication identifier, a product identifier, a medication schedule, a form of medication.

8. The computerized method of claim 7, wherein the set of product factors comprise: a medication identifier, a product identifier, a medication schedule form of medication, and a price.

9. The computerized method of claim 8, wherein the training data comprises a set of date characteristics comprising a calendar date the medication was dispensed, a national holiday schedule, a day of week, a day of the month, a month of the year, and a time of day.

10. The computerized method of claim 9, wherein the training data comprises a quantity of a medication identifier dispensed.

11. The computerized method of claim 10, wherein the training data comprises a pharmacy location where the medication is dispensed from.

12. The computerized method of claim 11, wherein the training data comprises an actual refill remaining on the prescription at a time of fill.

13. The computerized method of claim 12, wherein the training data comprises an expected number of refills remaining.

14. The computerized method of claim 13, wherein the training data comprises a set of factors affecting expected a general customer behavior.

15. The computer method of claim 14, wherein the general customer behavior comprises a holiday calendar schedule, a weather forecast, a month of year, and a day of week.

16. The computerized method of claim 15, wherein the training data comprises a set of factors affecting an expected individual customer behavior.

17. The computerize method of claim 16, wherein the expected individual customer behavior comprises an allergies state, an age variable, a gender variable, and a socioeconomic factors.

18. The computerized method of claim 17, wherein the training data comprises a set of historical inventory patterns by medication identifier.

19. The computerized method of claim 18, wherein the historical inventory patterns by medication identifier comprises an out-of-stock occurrences degree of overstocking expiration timeline.

20. A computer system for implementing machine learning model training and deployment for predictive inventory purchasing database comprising:
a processor;
a memory containing instructions when executed on the processor, causes the processor to perform operations that:
provide a predictive inventory purchasing database;
provide a machine-learning platform coupled with the predictive inventory purchasing database;
retrieve a set of training data as input to the machine learning platform;
based on the set of training data, generate a predictive inventory purchasing model with the machine learning platform; and
use the predictive inventory purchasing model to automatically implement inventory purchasing.
